(19) United States
(12) Patent Application Publication
(10) Pub. No.: US 2021/0161707 A1
(43) Pub. Date: Jun. 3, 2021

(54) DISPENSER FOR PRECISE EXTRACTION OF A SEMI-SOLID PRODUCT

(71) Applicant: Azura Ophthalmics Ltd., Tel Aviv (IL)

(72) Inventors: Yair ALSTER, Tel Aviv (IL); Omer RAFAELI, Udim (IL); Hadas Sarig RAPAPORT, Zofar (IL); Eitan SHARIFF, Gesher HaZiv (IL)

(21) Appl. No.: 17/171,942

(22) Filed: Feb. 9, 2021

Related U.S. Application Data
(63) Continuation of application No. PCT/IB2019/000903, filed on Aug. 9, 2019.
(60) Provisional application No. 62/717,197, filed on Aug. 10, 2018.

(51) Int. Cl. A61F 9/00; A61K 9/06; B65D 35/40; B65D 35/38; B65D 35/56

(52) U.S. Cl. CPC A61F 9/0026; A61K 9/06; B65D 35/56; B65D 35/38; B65D 35/40

(57) ABSTRACT

Provided herein are devices, systems, and methods for precise secretion of a semi-solid product from a product tube, which employ a visual indication corresponding to a secreted volume of the semi-solid product.

### CROSS-REFERENCE

This application is a continuation application of PCT/IB2019/000903, filed Aug. 9, 2019, and claims the benefit of U.S. Provisional Application No. 62/717,197, filed Aug. 10, 2018, all of which are incorporated herein by reference in their entirety.

### BACKGROUND OF THE INVENTION

A great variety of semi-solid drugs or cosmetics are currently being sold and tested for the treatment of a multitude of medical conditions or for cosmetic reasons. Such semi-solid may comprise products, creams, or gels. The use of such semi-solids to treat their respective medical conditions may comprise applying a set amount of the product to a specific portion of the body.

### SUMMARY OF THE INVENTION

One aspect provided herein is a dispenser for precise secretion of a semi-solid product from a product tube having a shoulder and a dispensing tip, the dispenser comprising: a collar comprising: an aperture configured to accept the dispensing tip; and a proximal surface configured to contact the shoulder of the product tube; and a first surface generally perpendicular to the proximal surface, wherein the first surface comprises at least one hyperacuity target extending from the proximal surface by a dispensing offset length; and wherein the at least one hyperacuity target provides a visual indication corresponding to the dispensing measurement distance and a secreted volume of the semi-solid product.

In some embodiments, the at least one hyperacuity target comprises a first hyperacuity target and a second hyperacuity target. In some embodiments, the first hyperacuity target and the second hyperacuity target further extend towards each other to form a hyperacuity target separation gap between a first proximal point on the first hyperacuity target and a second proximal point on the second hyperacuity target.

In some embodiments, the hyperacuity target separation gap is about 2 mm to about 60 mm. In some embodiments, the hyperacuity target separation gap is at least about 2 mm. In some embodiments, the hyperacuity target separation gap is at most about 60 mm. In some embodiments, the hyperacuity target separation gap is about 2 mm to about 4 mm, about 2 mm to about 8 mm, about 2 mm to about 10 mm, about 2 mm to about 15 mm, about 2 mm to about 20 mm, about 2 mm to about 30 mm, about 2 mm to about 40 mm, about 2 mm to about 50 mm, about 2 mm to about 60 mm, about 4 mm to about 8 mm, about 4 mm to about 10 mm, about 4 mm to about 15 mm, about 4 mm to about 20 mm, about 4 mm to about 30 mm, about 4 mm to about 40 mm, about 4 mm to about 50 mm, about 4 mm to about 60 mm, about 8 mm to about 10 mm, about 8 mm to about 15 mm, about 8 mm to about 20 mm, about 8 mm to about 30 mm, about 8 mm to about 40 mm, about 8 mm to about 50 mm, about 8 mm to about 60 mm, about 10 mm to about 15 mm, about 10 mm to about 20 mm, about 10 mm to about 30 mm, about 10 mm to about 40 mm, about 10 mm to about 50 mm, about 10 mm to about 60 mm, about 15 mm to about 20 mm, about 15 mm to about 30 mm, about 15 mm to about 40 mm, about 15 mm to about 50 mm, about 15 mm to about 60 mm, about 20 mm to about 30 mm, about 20 mm to about 40 mm, about 20 mm to about 50 mm, about 20 mm to about 60 mm, about 30 mm to about 40 mm, about 30 mm to about 50 mm, about 30 mm to about 60 mm, about 40 mm to about 50 mm, about 40 mm to about 60 mm, or about 50 mm to about 60 mm. In some embodiments, the hyperacuity target separation gap is about 2 mm, about 4 mm, about 8 mm, about 10 mm, about 15 mm, about 20 mm, about 30 mm, about 40 mm, about 50 mm, or about 60 mm.

In some embodiments, a distal edge of at least one of the first hyperacuity target and the second hyperacuity target are generally perpendicular to the proximal surface. In some embodiments, the distal edge of the first hyperacuity target and the distal edge of the second hyperacuity target are generally parallel or collinear. In some embodiments, the dispensing offset length is greater than a distance from the shoulder of the product tube to a distal point of the dispensing tip.

In some embodiments, the dispensing offset length is about 1 mm to about 100 mm. In some embodiments, the dispensing offset length is at least about 1 mm. In some embodiments, the dispensing offset length is at most about 100 mm. In some embodiments, the dispensing offset length is about 1 mm to about 5 mm, about 1 mm to about 10 mm, about 1 mm to about 20 mm, about 1 mm to about 30 mm, about 1 mm to about 40 mm, about 1 mm to about 50 mm, about 1 mm to about 60 mm, about 1 mm to about 70 mm, about 1 mm to about 80 mm, about 1 mm to about 90 mm, about 1 mm to about 100 mm, about 5 mm to about 10 mm, about 5 mm to about 20 mm, about 5 mm to about 30 mm, about 5 mm to about 40 mm, about 5 mm to about 50 mm, about 5 mm to about 60 mm, about 5 mm to about 70 mm, about 5 mm to about 80 mm, about 5 mm to about 90 mm, about 5 mm to about 100 mm, about 10 mm to about 20 mm, about 10 mm to about 30 mm, about 10 mm to about 40 mm, about 10 mm to about 50 mm, about 10 mm to about 60 mm, about 10 mm to about 70 mm, about 10 mm to about 80 mm, about 10 mm to about 90 mm, about 10 mm to about 100 mm, about 20 mm to about 30 mm, about 20 mm to about 40 mm, about 20 mm to about 50 mm, about 20 mm to about 60 mm, about 20 mm to about 70 mm, about 20 mm to about 80 mm, about 20 mm to about 90 mm, about 20 mm to about 100 mm, about 30 mm to about 40 mm, about 30 mm to about 50 mm, about 30 mm to about 60 mm, about 30 mm to about 70 mm, about 30 mm to about 80 mm, about 30 mm to about 90 mm, about 30 mm to about 100 mm, about 40 mm to about 50 mm, about 40 mm to about 60 mm, about 40 mm to about 70 mm, about 40 mm to about 80 mm, about 40 mm to about 90 mm, about 40 mm to about 100 mm, about 50 mm to about 60 mm, about 50 mm to about 70 mm, about 50 mm to about 80 mm, about 50 mm to about 90 mm, about 50 mm to about 100 mm, about 60 mm to about 70 mm, about 60 mm to about 80 mm, about 60 mm to about 90 mm, about 60 mm to about 100 mm, about 70 mm to about 80 mm, about 70 mm to about 90 mm, about 70 mm to about 100 mm, about 80 mm to about 90 mm, about 80 mm to about 100 mm, or about 90 mm to about 100 mm. In some embodiments, the dispensing offset length is about 1 mm, about 5 mm, about 10 mm, about 20 mm, about 30 mm, about 40 mm, about 50 mm, about 60 mm, about 70 mm, about 80 mm, about 90 mm, or about 100 mm.

In some embodiments, the product tube further comprises a cap, and wherein the aperture is further configured to accept the cap.

In some embodiments, the aperture has a diameter of about 3 mm to about 50 mm. In some embodiments, the aperture has a diameter of at least about 3 mm. In some embodiments, the aperture has a diameter of at most about 50 mm. In some embodiments, the aperture has a diameter of about 3 mm to about 4 mm, about 3 mm to about 5 mm, about 3 mm to about 6 mm, about 3 mm to about 8 mm, about 3 mm to about 10 mm, about 3 mm to about 20 mm, about 3 mm to about 30 mm, about 3 mm to about 40 mm, about 3 mm to about 50 mm, about 4 mm to about 5 mm, about 4 mm to about 6 mm, about 4 mm to about 8 mm, about 4 mm to about 10 mm, about 4 mm to about 20 mm, about 4 mm to about 30 mm, about 4 mm to about 40 mm, about 4 mm to about 50 mm, about 5 mm to about 6 mm, about 5 mm to about 8 mm, about 5 mm to about 10 mm, about 5 mm to about 20 mm, about 5 mm to about 30 mm, about 5 mm to about 40 mm, about 5 mm to about 50 mm, about 6 mm to about 8 mm, about 6 mm to about 10 mm, about 6 mm to about 20 mm, about 6 mm to about 30 mm, about 6 mm to about 40 mm, about 6 mm to about 50 mm, about 8 mm to about 10 mm, about 8 mm to about 20 mm, about 8 mm to about 30 mm, about 8 mm to about 40 mm, about 8 mm to about 50 mm, about 10 mm to about 20 mm, about 10 mm to about 30 mm, about 10 mm to about 40 mm, about 10 mm to about 50 mm, about 20 mm to about 30 mm, about 20 mm to about 40 mm, about 20 mm to about 50 mm, about 30 mm to about 40 mm, about 30 mm to about 50 mm, or about 40 mm to about 50 mm. In some embodiments, the aperture has a diameter of about 3 mm, about 4 mm, about 5 mm, about 6 mm, about 8 mm, about 10 mm, about 20 mm, about 30 mm, about 40 mm, or about 50 mm.

In some embodiments, the first surface further comprises a cavity configured surround at least a portion of the product tube. In some embodiments, the aperture of the collar comprises a threaded feature. In some embodiments, the dispenser comprises plastic, wood, metal, fiberglass, carbon fiber, resin, or any combination thereof.

Another aspect provided herein is a product tube for precise secretion of a semi-solid product comprising: a product tube body configured to contain the semi-solid product; a nozzle configured to secrete the semi-solid product from the product tube body; and at least one hyperacuity target extending beyond the nozzle by a dispensing measurement distance. In some embodiments, the at least one hyperacuity target provides a visual indication corresponding to the dispensing measurement distance and a secreted volume of the semi-solid product.

In some embodiments, the at least one hyperacuity target comprises a first hyperacuity target and a second hyperacuity target.

In some embodiments, the secreted volume is about 1 ul (microliter) to about 10 ul. In some embodiments, the secreted volume is at least about 1 ul. In some embodiments, the secreted volume is at most about 10 ul. In some embodiments, the secreted volume is about 1 ul to about 2 ul, about 1 ul to about 3 ul, about 1 ul to about 4 ul, about 1 ul to about 5 ul, about 1 ul to about 6 ul, about 1 ul to about 7 ul, about 1 ul to about 8 ul, about 1 ul to about 9 ul, about 1 ul to about 10 ul, about 2 ul to about 3 ul, about 2 ul to about 4 ul, about 2 ul to about 5 ul, about 2 ul to about 6 ul, about 2 ul to about 7 ul, about 2 ul to about 8 ul, about 2 ul to about 9 ul, about 2 ul to about 10 ul, about 3 ul to about 4 ul, about 3 ul to about 5 ul, about 3 ul to about 6 ul, about 3 ul to about 7 ul, about 3 ul to about 8 ul, about 3 ul to about 9 ul, about 3 ul to about 10 ul, about 4 ul to about 5 ul, about 4 ul to about 6 ul, about 4 ul to about 7 ul, about 4 ul to about 8 ul, about 4 ul to about 9 ul, about 4 ul to about 10 ul, about 5 ul to about 6 ul, about 5 ul to about 7 ul, about 5 ul to about 8 ul, about 5 ul to about 9 ul, about 5 ul to about 10 ul, about 6 ul to about 7 ul, about 6 ul to about 8 ul, about 6 ul to about 9 ul, about 6 ul to about 10 ul, about 7 ul to about 8 ul, about 7 ul to about 9 ul, about 7 ul to about 10 ul, about 8 ul to about 9 ul, about 8 ul to about 10 ul, or about 9 ul to about 10 ul. In some embodiments, the secreted volume is about 1 ul, about 2 ul, about 3 ul, about 4 ul, about 5 ul, about 6 ul, about 7 ul, about 8 ul, about 9 ul, or about 10 ul.

In some embodiments, the secreted amount is about 1 mg (milligram) to about 10 mg. In some embodiments, the secreted amount is at least about 1 mg. In some embodiments, the secreted amount is at most about 10 mg. In some embodiments, the secreted amount is about 1 mg to about 2 mg, about 1 mg to about 3 mg, about 1 mg to about 4 mg, about 1 mg to about 5 mg, about 1 mg to about 6 mg, about 1 mg to about 7 mg, about 1 mg to about 8 mg, about 1 mg to about 9 mg, about 1 mg to about 10 mg, about 2 mg to about 3 mg, about 2 mg to about 4 mg, about 2 mg to about 5 mg, about 2 mg to about 6 mg, about 2 mg to about 7 mg, about 2 mg to about 8 mg, about 2 mg to about 9 mg, about 2 mg to about 10 mg, about 3 mg to about 4 mg, about 3 mg to about 5 mg, about 3 mg to about 6 mg, about 3 mg to about 7 mg, about 3 mg to about 8 mg, about 3 mg to about 9 mg, about 3 mg to about 10 mg, about 4 mg to about 5 mg, about 4 mg to about 6 mg, about 4 mg to about 7 mg, about 4 mg to about 8 mg, about 4 mg to about 9 mg, about 4 mg to about 10 mg, about 5 mg to about 6 mg, about 5 mg to about 7 mg, about 5 mg to about 8 mg, about 5 mg to about 9 mg, about 5 mg to about 10 mg, about 6 mg to about 7 mg, about 6 mg to about 8 mg, about 6 mg to about 9 mg, about 6 mg to about 10 mg, about 7 mg to about 8 mg, about 7 mg to about 9 mg, about 7 mg to about 10 mg, about 8 mg to about 9 mg, about 8 mg to about 10 mg, or about 9 mg to about 10 mg. In some embodiments, the secreted amount is about 1 mg, about 2 mg, about 3 mg, about 4 mg, about 5 mg, about 6 mg, about 7 mg, about 8 mg, about 9 mg, or about 10 mg.

In some embodiments, an accuracy of the secreted volume is about 0.1 ul to about 10 ul. In some embodiments, the accuracy is at least about 0.1 ul. In some embodiments, the accuracy is at most about 10 ul. In some embodiments, the accuracy is about 0.1 ul to about 0.2 ul, about 0.1 ul to about 0.5 ul, about 0.1 ul to about 1 ul, about 0.1 ul to about 2 ul, about 0.1 ul to about 3 ul, about 0.1 ul to about 4 ul, about 0.1 ul to about 5 ul, about 0.1 ul to about 6 ul, about 0.1 ul to about 7 ul, about 0.1 ul to about 8 ul, about 0.1 ul to about 10 ul, about 0.2 ul to about 0.5 ul, about 0.2 ul to about 1 ul, about 0.2 ul to about 2 ul, about 0.2 ul to about 3 ul, about 0.2 ul to about 4 ul, about 0.2 ul to about 5 ul, about 0.2 ul to about 6 ul, about 0.2 ul to about 7 ul, about 0.2 ul to about 8 ul, about 0.2 ul to about 10 ul, about 0.5 ul to about 1 ul, about 0.5 ul to about 2 ul, about 0.5 ul to about 3 ul, about 0.5 ul to about 4 ul, about 0.5 ul to about 5 ul, about 0.5 ul to about 6 ul, about 0.5 ul to about 7 ul, about 0.5 ul to about 8 ul, about 0.5 ul to about 10 ul, about 1 ul to about 2 ul, about 1 ul to about 3 ul, about 1 ul to about 4 ul, about 1 ul to about 5 ul, about 1 ul to about 6 ul, about 1 ul to about 7 ul, about 1 ul to about 8 ul, about 1 ul to about 10 ul, about 2 ul to about 3 ul, about 2 ul to about 4 ul, about 2 ul to about 5 ul, about 2 ul to about 6 ul, about 2 ul to about 7 ul, about 2 ul to about 8 ul, about 2 ul to about 10 ul, about 3 ul to about 4 ul, about 3 ul to about 5 ul, about 3 ul to about 6 ul, about 3 ul to about 7 ul, about 3 ul to about 8 ul, about 3 ul to about 10 ul, about 4 ul to about 5 ul, about 4 ul to about 6 ul, about 4 ul to about 7 ul, about 4 ul to about 8 ul, about 4 ul to about 10 ul, about 5 ul to about 6 ul, about 5 ul to about 7 ul, about 5 ul to about 8 ul, about 5 ul to about 10 ul, about 6 ul to about 7 ul, about 6 ul to about 8 ul, about 6 ul to about 10 ul, about 7 ul to about 8 ul, about 7 ul to about 10 ul, or about 8 ul to about 10 ul. In some embodiments, the accuracy is about 0.1 ul, about 0.2 ul, about 0.5 ul, about 1 ul, about 2 ul, about 3 ul, about 4 ul, about 5 ul, about 6 ul, about 7 ul, about 8 ul, or about 10 ul.

In some embodiments, the first hyperacuity target and the second hyperacuity target further extend towards each other to form a hyperacuity target separation gap between a first proximal point on the first hyperacuity target and a second proximal point on the second hyperacuity target. In some embodiments, the hyperacuity target separation gap is about 2 mm to about 60 mm. In some embodiments, the hyperacuity target separation gap is at least about 2 mm. In some embodiments, the distal edge of the first hyperacuity target and the distal edge of the second hyperacuity target are generally parallel or collinear.

In some embodiments, the dispensing measurement distance is about 1 mm to about 15 mm. In some embodiments, the dispensing measurement distance is at least about 1 mm. In some embodiments, the dispensing measurement distance is at most about 15 mm. In some embodiments, the dispensing measurement distance is about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 10 mm, about 1 mm to about 12 mm, about 1 mm to about 15 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 10 mm, about 2 mm to about 12 mm, about 2 mm to about 15 mm, about 3 mm to about 4 mm, about 3 mm to about 5 mm, about 3 mm to about 6 mm, about 3 mm to about 7 mm, about 3 mm to about 8 mm, about 3 mm to about 10 mm, about 3 mm to about 12 mm, about 3 mm to about 15 mm, about 4 mm to about 5 mm, about 4 mm to about 6 mm, about 4 mm to about 7 mm, about 4 mm to about 8 mm, about 4 mm to about 10 mm, about 4 mm to about 12 mm, about 4 mm to about 15 mm, about 5 mm to about 6 mm, about 5 mm to about 7 mm, about 5 mm to about 8 mm, about 5 mm to about 10 mm, about 5 mm to about 12 mm, about 5 mm to about 15 mm, about 6 mm to about 7 mm, about 6 mm to about 8 mm, about 6 mm to about 10 mm, about 6 mm to about 12 mm, about 6 mm to about 15 mm, about 7 mm to about 8 mm, about 7 mm to about 10 mm, about 7 mm to about 12 mm, about 7 mm to about 15 mm, about 8 mm to about 10 mm, about 8 mm to about 12 mm, about 8 mm to about 15 mm, about 10 mm to about 12 mm, about 10 mm to about 15 mm, or about 12 mm to about 15 mm. In some embodiments, the dispensing measurement distance is about 1 mm, about 2 mm, about 3 mm, about 4 mm, about 5 mm, about 6 mm, about 7 mm, about 8 mm, about 10 mm, about 12 mm, or about 15 mm.

In some embodiments, the product tube comprises comprising plastic, wood, metal, fiberglass, carbon fiber, resin, or any combination thereof.

Another aspect provided herein is a system for precise secretion of a semi-solid product comprising: a product tube having a shoulder and a dispensing tip; and a dispenser comprising: a collar comprising: an aperture configured to accept the dispensing tip; a proximal surface configured to contact the shoulder of the product tube; and a first surface generally perpendicular to the proximal surface, wherein the first surface comprises at least one hyperacuity target extending from the proximal surface by a dispensing measurement distance; wherein at least one of the first hyperacuity target and the second hyperacuity target provide a visual indication corresponding to the dispensing measurement distance and a secreted volume of the semi-solid product.

In some embodiments, the at least one hyperacuity target comprises a first hyperacuity target and a second hyperacuity target.

In some embodiments, the secreted volume is about 1 ul (microliter) to about 10 ul. In some embodiments, the secreted volume is at least about 1 ul. In some embodiments, the secreted volume is at most about 10 ul. In some embodiments, the secreted volume is about 1 ul to about 2 ul, about 1 ul to about 3 ul, about 1 ul to about 4 ul, about 1 ul to about 5 ul, about 1 ul to about 6 ul, about 1 ul to about 7 ul, about 1 ul to about 8 ul, about 1 ul to about 9 ul, about 1 ul to about 10 ul, about 2 ul to about 3 ul, about 2 ul to about 4 ul, about 2 ul to about 5 ul, about 2 ul to about 6 ul, about 2 ul to about 7 ul, about 2 ul to about 8 ul, about 2 ul to about 9 ul, about 2 ul to about 10 ul, about 3 ul to about 4 ul, about 3 ul to about 5 ul, about 3 ul to about 6 ul, about 3 ul to about 7 ul, about 3 ul to about 8 ul, about 3 ul to about 9 ul, about 3 ul to about 10 ul, about 4 ul to about 5 ul, about 4 ul to about 6 ul, about 4 ul to about 7 ul, about 4 ul to about 8 ul, about 4 ul to about 9 ul, about 4 ul to about 10 ul, about 5 ul to about 6 ul, about 5 ul to about 7 ul, about 5 ul to about 8 ul, about 5 ul to about 9 ul, about 5 ul to about 10 ul, about 6 ul to about 7 ul, about 6 ul to about 8 ul, about 6 ul to about 9 ul, about 6 ul to about 10 ul, about 7 ul to about 8 ul, about 7 ul to about 9 ul, about 7 ul to about 10 ul, about 8 ul to about 9 ul, about 8 ul to about 10 ul, or about 9 ul to about 10 ul. In some embodiments, the secreted volume is about 1 ul, about 2 ul, about 3 ul, about 4 ul, about 5 ul, about 6 ul, about 7 ul, about 8 ul, about 9 ul, or about 10 ul.

In some embodiments, the secreted amount is about 1 mg (milligram) to about 10 mg. In some embodiments, the secreted amount is at least about 1 mg. In some embodiments, the secreted amount is at most about 10 mg. In some embodiments, the secreted amount is about 1 mg to about 2 mg, about 1 mg to about 3 mg, about 1 mg to about 4 mg, about 1 mg to about 5 mg, about 1 mg to about 6 mg, about 1 mg to about 7 mg, about 1 mg to about 8 mg, about 1 mg to about 9 mg, about 1 mg to about 10 mg, about 2 mg to about 3 mg, about 2 mg to about 4 mg, about 2 mg to about 5 mg, about 2 mg to about 6 mg, about 2 mg to about 7 mg, about 2 mg to about 8 mg, about 2 mg to about 9 mg, about 2 mg to about 10 mg, about 3 mg to about 4 mg, about 3 mg to about 5 mg, about 3 mg to about 6 mg, about 3 mg to about 7 mg, about 3 mg to about 8 mg, about 3 mg to about 9 mg, about 3 mg to about 10 mg, about 4 mg to about 5 mg, about 4 mg to about 6 mg, about 4 mg to about 7 mg, about 4 mg to about 8 mg, about 4 mg to about 9 mg, about 4 mg to about 10 mg, about 5 mg to about 6 mg, about 5 mg to about 7 mg, about 5 mg to about 8 mg, about 5 mg to about 9 mg, about 5 mg to about 10 mg, about 6 mg to about 7 mg, about 6 mg to about 8 mg, about 6 mg to about 9 mg, about 6 mg to about 10 mg, about 7 mg to about 8 mg, about 7 mg to about 9 mg, about 7 mg to about 10 mg, about 8 mg to about 9 mg, about 8 mg to about 10 mg, or about 9 mg to about 10 mg. In some embodiments, the secreted amount is about 1 mg, about 2 mg, about 3 mg, about 4 mg, about 5 mg, about 6 mg, about 7 mg, about 8 mg, about 9 mg, or about 10 mg.

In some embodiments, an accuracy of the secreted volume is about 0.1 ul to about 10 ul. In some embodiments, the accuracy is at least about 0.1 ul. In some embodiments, the accuracy is at most about 10 ul. In some embodiments, the accuracy is about 0.1 ul to about 0.2 ul, about 0.1 ul to about 0.5 ul, about 0.1 ul to about 1 ul, about 0.1 ul to about 2 ul, about 0.1 ul to about 3 ul, about 0.1 ul to about 4 ul, about 0.1 ul to about 5 ul, about 0.1 ul to about 6 ul, about 0.1 ul to about 7 ul, about 0.1 ul to about 8 ul, about 0.1 ul to about 10 ul, about 0.2 ul to about 0.5 ul, about 0.2 ul to about 1 ul, about 0.2 ul to about 2 ul, about 0.2 ul to about 3 ul, about 0.2 ul to about 4 ul, about 0.2 ul to about 5 ul, about 0.2 ul to about 6 ul, about 0.2 ul to about 7 ul, about 0.2 ul to about 8 ul, about 0.2 ul to about 10 ul, about 0.5 ul to about 1 ul, about 0.5 ul to about 2 ul, about 0.5 ul to about 3 ul, about 0.5 ul to about 4 ul, about 0.5 ul to about 5 ul, about 0.5 ul to about 6 ul, about 0.5 ul to about 7 ul, about 0.5 ul to about 8 ul, about 0.5 ul to about 10 ul, about 1 ul to about 2 ul, about 1 ul to about 3 ul, about 1 ul to about 4 ul, about 1 ul to about 5 ul, about 1 ul to about 6 ul, about 1 ul to about 7 ul, about 1 ul to about 8 ul, about 1 ul to about 10 ul, about 2 ul to about 3 ul, about 2 ul to about 4 ul, about 2 ul to about 5 ul, about 2 ul to about 6 ul, about 2 ul to about 7 ul, about 2 ul to about 8 ul, about 2 ul to about 10 ul, about 3 ul to about 4 ul, about 3 ul to about 5 ul, about 3 ul to about 6 ul, about 3 ul to about 7 ul, about 3 ul to about 8 ul, about 3 ul to about 10 ul, about 4 ul to about 5 ul, about 4 ul to about 6 ul, about 4 ul to about 7 ul, about 4 ul to about 8 ul, about 4 ul to about 10 ul, about 5 ul to about 6 ul, about 5 ul to about 7 ul, about 5 ul to about 8 ul, about 5 ul to about 10 ul, about 6 ul to about 7 ul, about 6 ul to about 8 ul, about 6 ul to about 10 ul, about 7 ul to about 8 ul, about 7 ul to about 10 ul, or about 8 ul to about 10 ul. In some embodiments, the accuracy is about 0.1 ul, about 0.2 ul, about 0.5 ul, about 1 ul, about 2 ul, about 3 ul, about 4 ul, about 5 ul, about 6 ul, about 7 ul, about 8 ul, or about 10 ul.

In some embodiments, the first hyperacuity target and the second hyperacuity target further extend towards each other to form a hyperacuity target separation gap between a first proximal point on the first hyperacuity target and a second proximal point on the second hyperacuity target. In some embodiments, the hyperacuity target separation gap is about 2 mm to about 60 mm. In some embodiments, the hyperacuity target separation gap is at least about 2 mm. In some embodiments, a distal edge of at least one of the first hyperacuity target and the second hyperacuity target are generally perpendicular to the proximal surface. In some embodiments, the distal edge of the first hyperacuity target and the distal edge of the second hyperacuity target are generally parallel or collinear. In some embodiments, the dispensing measurement distance is a greater than a distance from the shoulder of the semi-solid product tube to a distal point of the dispensing tip.

In some embodiments, the dispensing measurement distance is about 1 mm to about 15 mm. In some embodiments, the dispensing measurement distance is at least about 1 mm. In some embodiments, the dispensing measurement distance is at most about 15 mm. In some embodiments, the dispensing measurement distance is about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 10 mm, about 1 mm to about 12 mm, about 1 mm to about 15 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 10 mm, about 2 mm to about 12 mm, about 2 mm to about 15 mm, about 3 mm to about 4 mm, about 3 mm to about 5 mm, about 3 mm to about 6 mm, about 3 mm to about 7 mm, about 3 mm to about 8 mm, about 3 mm to about 10 mm, about 3 mm to about 12 mm, about 3 mm to about 15 mm, about 4 mm to about 5 mm, about 4 mm to about 6 mm, about 4 mm to about 7 mm, about 4 mm to about 8 mm, about 4 mm to about 10 mm, about 4 mm to about 12 mm, about 4 mm to about 15 mm, about 5 mm to about 6 mm, about 5 mm to about 7 mm, about 5 mm to about 8 mm, about 5 mm to about 10 mm, about 5 mm to about 12 mm, about 5 mm to about 15 mm, about 6 mm to about 7 mm, about 6 mm to about 8 mm, about 6 mm to about 10 mm, about 6 mm to about 12 mm, about 6 mm to about 15 mm, about 7 mm to about 8 mm, about 7 mm to about 10 mm, about 7 mm to about 12 mm, about 7 mm to about 15 mm, about 8 mm to about 10 mm, about 8 mm to about 12 mm, about 8 mm to about 15 mm, about 10 mm to about 12 mm, about 10 mm to about 15 mm, or about 12 mm to about 15 mm. In some embodiments, the dispensing measurement distance is about 1 mm, about 2 mm, about 3 mm, about 4 mm, about 5 mm, about 6 mm, about 7 mm, about 8 mm, about 10 mm, about 12 mm, or about 15 mm.

In some embodiments, the product tube further comprises a cap, and wherein the aperture is further configured to accept the cap. In some embodiments, the aperture has a diameter of about 3 mm to about 50 mm. In some embodiments, the aperture has a diameter of at least about 3 mm. In some embodiments, the first surface further comprises a cavity configured surround at least a portion of the product tube. In some embodiments, the aperture of the collar comprises a threaded feature. In some embodiments, the dispenser comprises plastic, wood, metal, fiberglass, carbon fiber, resin, or any combination thereof.

Another aspect provided herein is a kit comprising: two or more product tubes containing a semi-solid product, each product tube having a semi-solid product; and a dispenser. In some embodiments, the kit further comprises at least one of an applicator, an instruction manual, a mirror, and a wipe.

Another aspect provided herein is a method for precise secretion of a semi-solid product comprising: providing a product tube having a shoulder and a dispensing tip; providing a dispenser; aligning the shoulder of the product tube against the proximal surface of the dispenser; and compressing the product tube to secrete the semi-solid product until the semi-solid product is level with a proximal portion of the first hyperacuity target, a proximal portion of the second hyperacuity target, or both. In some embodiments, the method further comprises removing the secreted semi-solid product from the product tube. In some embodiments, removing the secreted semi-solid product from the product tube comprises removing the secreted semi-solid product from the product tube with an applicator or a finger. In some embodiments, the method further comprises applying the semi-solid product to a patient. In some embodiments, the product tube further comprises a cap, and wherein the method further comprises removing the cap. In some embodiments, the cap is removed before inserting the dispensing tip of the product tube within the aperture of the dispenser. In some embodiments, the cap is removed after inserting the dispensing tip of the product tube within the aperture of the dispenser. In some embodiments, the method further comprises replacing the cap onto the product tube after removing the secreted semi-solid product from the product tube.

Another aspect provided herein is a method for precise secretion of a semi-solid product comprising: providing a product tube; and compressing the product tube to secrete the semi-solid product until the semi-solid product is level with a proximal portion of the first hyperacuity target, a proximal portion of the second hyperacuity target, or both.

In some embodiments, the method further comprises removing the secreted semi-solid product from the product tube. In some embodiments, removing the secreted semi-solid product from the product tube comprises removing the secreted semi-solid product from the product tube with an applicator or a finger. In some embodiments, the method further comprises applying the semi-solid to a patient. In some embodiments, the product tube further comprises a cap, and wherein the method further comprises removing the cap. In some embodiments, the method further comprises replacing the cap onto the product tube after removing the secreted semi-solid product from the product tube.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a front view illustration of an exemplary (e.g., first) dispenser, per some embodiments herein;

FIG. 2 is a top view illustration of an exemplary (e.g., first) dispenser, per some embodiments herein;

FIG. 3 is a front view illustration of an exemplary (e.g., second) dispenser(e.g., comprising a recess), per some embodiments herein;

FIG. 4 is a front view illustration of an exemplary (e.g., first) semi solid product tube, per some embodiments herein;

FIG. 5 is a front view illustration of an exemplary (e.g., first) system comprising a semi solid product dispenser and a product tube, per some embodiments herein;

FIG. 6 is an image of an exemplary (e.g., first) system comprising a semi solid product dispenser and a product tube, per some embodiments herein;

FIG. 7 is another image of an exemplary (e.g., first) system comprising a semi solid product dispenser and a product tube, per some embodiments herein;

FIG. 8 is an image of an exemplary (e.g., first) system comprising a semi solid product dispenser and a product tube, per some embodiments herein;

FIG. 9 is a front view illustration of an exemplary (e.g., second) dispenser, per some embodiments herein;

FIG. 10 is a perspective view illustration of an exemplary (e.g., second) dispenser, per some embodiments herein;

FIG. 11 is a perspective view illustration of an exemplary (e.g., second) dispenser, per some embodiments herein;

FIG. 12 is a top view illustration of an exemplary (e.g., second) dispenser, per some embodiments herein;

FIG. 13 is a side view illustration of an exemplary (e.g., second) dispenser, per some embodiments herein;

FIG. 14 is a front view illustration of an exemplary (e.g., third) dispenser, per some embodiments herein;

FIG. 15 is a front view illustration of an exemplary (e.g., fourth) dispenser, per some embodiments herein;

FIG. 16 is a front view illustration of an exemplary (e.g., second) semi solid product tube, per some embodiments herein;

FIG. 17 is an exploded front view illustration of the exemplary (e.g., second) semi solid product tube comprising a cap, per some embodiments herein;

FIG. 18 is a collapsed front view illustration of the exemplary (e.g., second) semi solid product tube, per some embodiments herein;

FIG. 19 is a top view illustration of the exemplary (e.g., second) semi solid product tube, per some embodiments herein;

FIG. 20 is a side view illustration of the exemplary (e.g., second) semi solid product tube, per some embodiments herein;

FIG. 21 is a top view illustration of an exemplary (e.g., third) semi solid product tube, per some embodiments herein;

FIG. 22 is a top view illustration of an exemplary (e.g., fourth) semi solid product tube, per some embodiments herein;

FIG. 23 is a front view illustration of an exemplary (e.g., first) system comprising a semi solid product dispenser and a product tube, per some embodiments herein;

FIG. 24 is a front exploded view illustration of an exemplary (e.g., first) system, per some embodiments herein;

FIG. 25 is a front collapsed view illustration of an exemplary (e.g., first) system, per some embodiments herein;

FIG. 26 is a front view illustration of an exemplary (e.g., second) system comprising a semi solid product dispenser and a product tube, per some embodiments herein; and

FIG. 27 is a front view illustration of an exemplary (e.g., third) system comprising a semi solid product dispenser and a product tube, per some embodiments herein.

### DETAILED DESCRIPTION OF THE INVENTION

While eye drops have been used to dispense controlled liquid volumes of a medicament, such devices are incapable of properly dispensing semi-solid drugs or cosmetic products. Further, such eye drops exhibit poor accuracies, whereby the liquid volume per drop may range from 30-50 microliters, and whereby the number of dispensed drops cannot be easily controlled. Additionally, inhalers and syringes have been developed for dosing aqueous medicaments through controlled dosing orifice sizes and timed control. Such solutions, however, may not be configured for proper dispensing of semi-solid products, gels, ointments, or creams.

Accurate dosing of small amounts of semi-solid products has been difficult to achieve due to the high viscosity and non-Newtonian aspects of such semi-solids. Further, as such dosing mechanisms are currently large and expensive, their use for the treatment of temporary conditions, or for short-term clinical trials are limited. Excessive application of many semi-solid products may cause unwanted side-effects. For example, excessive application of eye products may blur vision by creating thick layers that mixes into the tear film. Insufficient application of many products may slow or prevent proper treatment. Additionally, controlled dispensed volumes may further improve the application accuracy for proper treatment. For example, excessive eye medicament applied to the eye may be accidentally applied to the eye-lid margin. Finally, many such medicaments are sensitive to dirt, particulate, and oils, and must be transferred directly to the treatment site without contact with non-applicator surfaces, to avoid contamination.

Many semi-solid products are stored and dispensed from product tubes, which are often improper for accurate dosing. As such product tubes may be formed of metal, plastic, or both, the internal dispensing pressure may continue or reverse once the force applied by the user terminates.

As such, provided herein are intuitive, cost-effective dispensing aids, systems, and method which enable accurately control the semi-solid product dosing.

Dispenser for precise secretion of a semi-solid product from a product tube

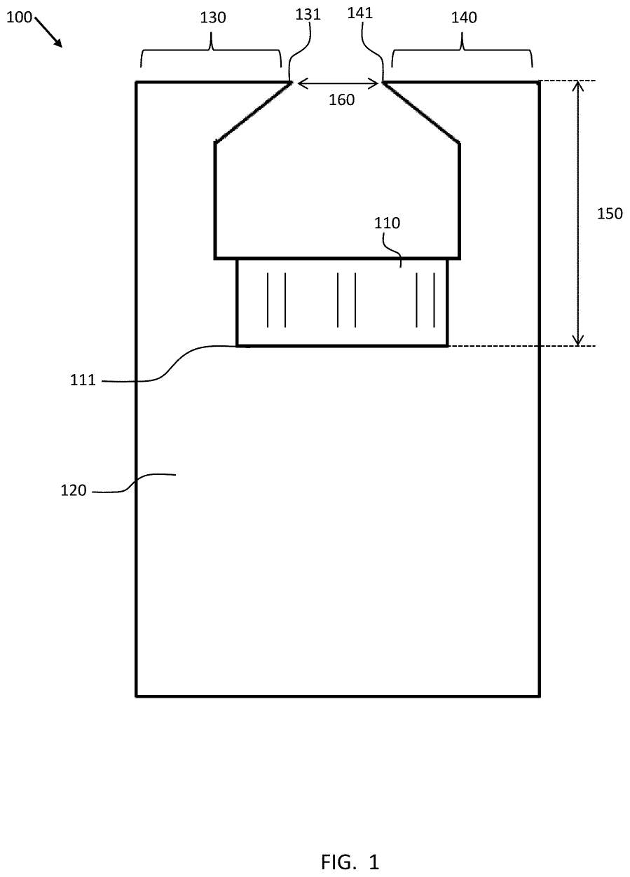

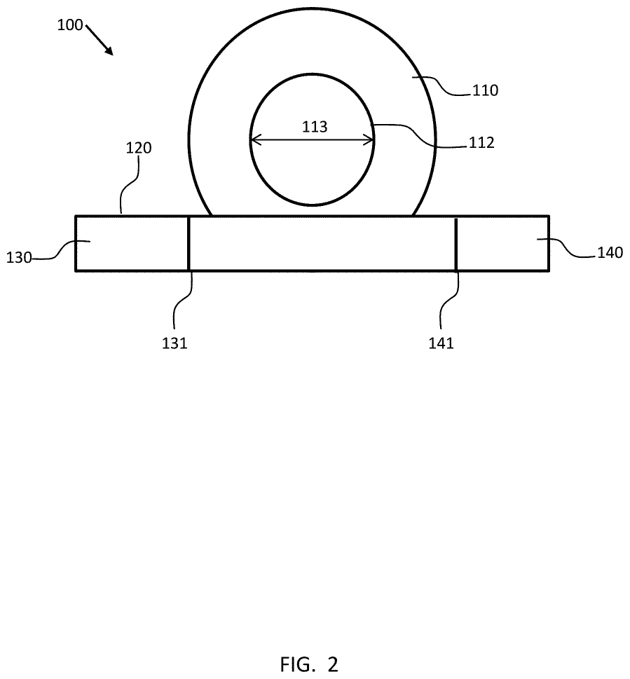

Described herein, per FIGS. 1-3, is an exemplary (e.g., first) dispenser 100 for precise secretion of a semi-solid product from a product tube (not shown). In various embodiments, the semi-solid is an ointment, a cream, or a gel, or other suitable formulation.

In some embodiments, the product tube has a shoulder and a dispensing tip. In some embodiments, the product tube further comprises a cap.

The first dispenser 100 may comprise a collar 110 and a first surface 120. The collar 110 may comprise an aperture 112 and a proximal surface 111 (e.g., proximal to a receiving end of aperture 112, such as for receiving a product tube, or tip thereof, therethrough). The aperture 112 may be configured to accept the dispensing tip (e.g., through the receiving end thereof, such as configured proximal to the proximal surface 111). The aperture 112 may be configured to accept the cap. The proximal surface 111 may be configured to contact the shoulder of the product tube. In some embodiments, the aperture 112 has a diameter 113 of about 3 mm to about 50 mm. In some embodiments, the aperture 112 has a diameter 113 of at least about 3 mm. The aperture diameter 113 may be measured as a minimum aperture diameter 113, or an average aperture diameter 113. In some embodiments, the aperture 112 of the collar 110 comprises a threaded feature. In some embodiments, the first dispenser 100 comprises plastic, wood, metal, fiberglass, carbon fiber, resin, or any combination thereof.

In certain embodiments, a dispenser provided herein comprises a first surface 120, such as wherein the first surface 120 may be generally perpendicular to the proximal surface 111. The first surface 120 may comprise at least one hyperacuity target 130, 140. The at least one hyperacuity target 130, 140 may comprise a first hyperacuity target 130 and a second hyperacuity target 140. In some embodiments, a distal edge of at least one of the first hyperacuity target 130 and the second hyperacuity target 140 are generally perpendicular to the proximal surface 111. In some embodiments, the distal edge of the first hyperacuity target 130 and the distal edge of the second hyperacuity target 140 are generally parallel or collinear. At least one of the first hyperacuity target 130 and the second hyperacuity target 140 may provide a visual indication corresponding to the dispensing measurement distance and a secreted volume of the semi-solid product. At least one of the first hyperacuity target 130 and the second hyperacuity target 140 may provide a visual indication corresponding to the dispensing measurement distance and a secreted amount or dosage of the semi-solid product. The at least one hyperacuity target 130, 140 may terminate at a set distance from the nozzle of the product tube to prevent contact between the at least one hyperacuity target 130, 140 and the semi-solid product. Preventing contact may enable sterility of the semi-solid product.

The visual indication may comprise a hyperacuity visual indication, wherein a human eye automatically employs Vernier acuity to identify a very small misalignment between two or more visual stimuli (such as two lines segments). This Vernier acuity of the human eye has been shown to be able to discriminate misalignment of 3-6 sec arc. As this phenomenon enables sensitivity of an order of magnitude greater than visual acuity (the smallest number or letter human eye can recognize) hyperacuity may be implemented to enable simple and precise alignment measurements.

In some embodiments, the first hyperacuity target 130 and the second hyperacuity target 140 further extend towards each other. In some embodiments, the first hyperacuity target 130 and the second hyperacuity target 140 further extend towards each other to form a hyperacuity target separation gap 160. The separation gap 160 may be measured between a first proximal point and a second proximal point. The separation gap 160 may be measured as a minimum distance between the first proximal point 131 on the first hyperacuity target 130 and the second proximal point 141 on the second hyperacuity target 140. The separation gap 160 may be measured as a minimum normal distance between the first proximal point 131 on the first hyperacuity target 130 and the second proximal point 141 on the second hyperacuity target 140. The separation gap 160 may be measured as a minimum normal distance between the first hyperacuity target 130 and the second hyperacuity target 140. In some embodiments, the hyperacuity target separation gap 160 is about 2 mm to about 60 mm. In some embodiments, the hyperacuity target separation gap 160 is at least about 2 mm. The separation gap may be designed to prevent contact between the first hyperacuity target 130 and the semi-solid product, the second hyperacuity target 140 and the semi-solid product, or both. Preventing contact may enable sterility of the semi-solid product.

The first hyperacuity target 130 may extend from the proximal surface 111 by a dispensing offset length 150. The second hyperacuity target 140 may extend from the proximal surface 111 by the dispensing offset length 150. In some embodiments, the dispensing offset length 150 is a greater than a distance from the shoulder of the product tube to a distal point of the dispensing tip. In some embodiments, the dispensing offset length 150 is about 1 mm to about 100 mm. In some embodiments, the dispensing offset length 150 is at least about 1 mm. The dispensing offset length 150 may be measured as a maximum distance between the proximal surface 111 and a first distal point on the first hyperacuity target 130 or a second distal point on the second hyperacuity target 140. The dispensing offset length 150 may be measured as a maximum normal distance between the proximal surface 111 and the first distal point on the first hyperacuity target 130 or the second distal point on the second hyperacuity target 140. In some embodiments, the first distal point is the first proximal point 131. In some embodiments, the second distal point is the second proximal point 141. In some embodiments, the dispensing offset length 150 corresponds to the secreted volume of the semi-solid product. In some embodiments, the secreted volume is about 1 ul to about 10 ul. In some embodiments, the secreted volume is about 1 mg to about 10 mg. In some embodiments, the secreted volume accuracy is about 0.1 ul to about 10 ul. In some embodiments, the secreted volume corresponds to a viscosity, a melting temperature, a kinematic viscosity, or any combination thereof of the semi-solid product.

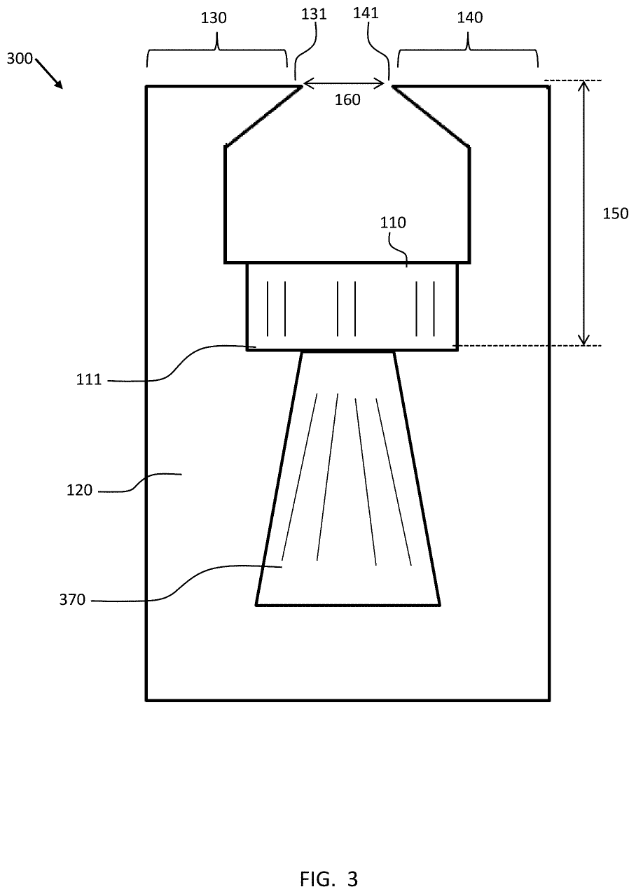

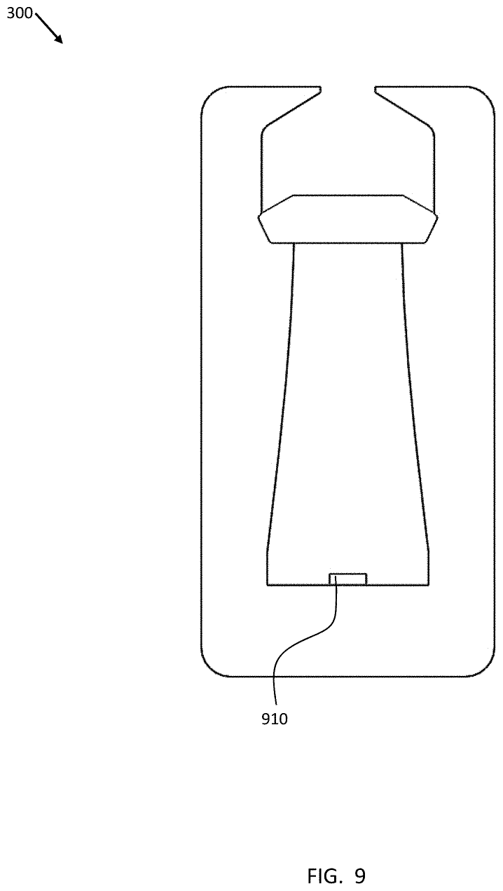

An exemplary second dispenser 300 is shown in FIG. 3, wherein the first surface 120 further comprises a recess 370 configured to receive and/or surround at least a portion of the product tube. As shown, in some instances, the recess 370 is tapered. Alternatively, in some embodiments, the recess 370 has a planar shape comprising a circle, a square, or any polygon. Various embodiments of the dispenser 300 is further illustrated in FIGS. 9-13 wherein dispenser 300 comprises a tube fastener 910. As shown, the tube fastener 910 comprises a ledge. Alternatively in some embodiments, the tube fastener 910 comprises a clip, a channel, or any other element configured to releasably constrain a product tube.

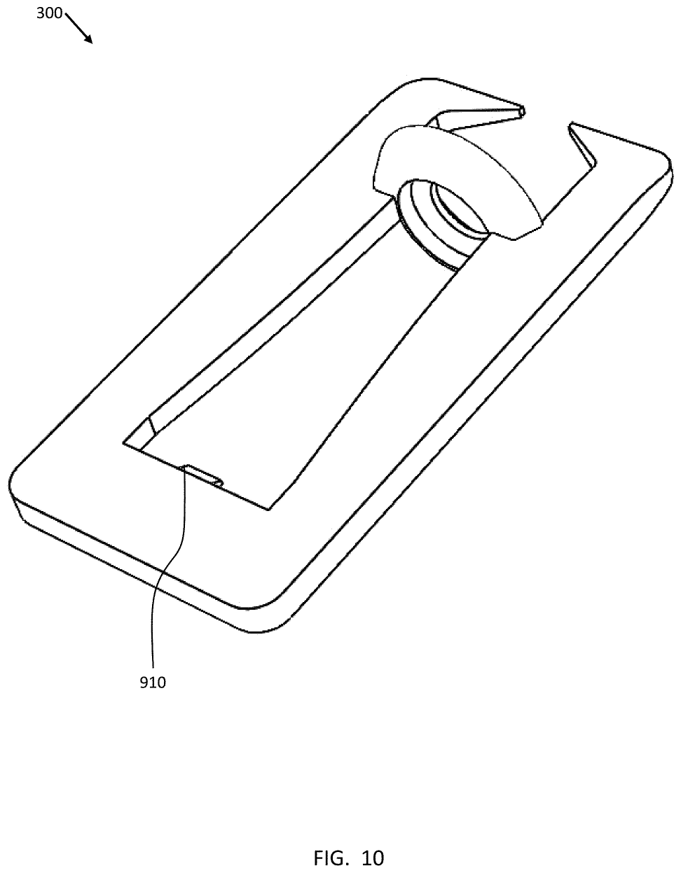

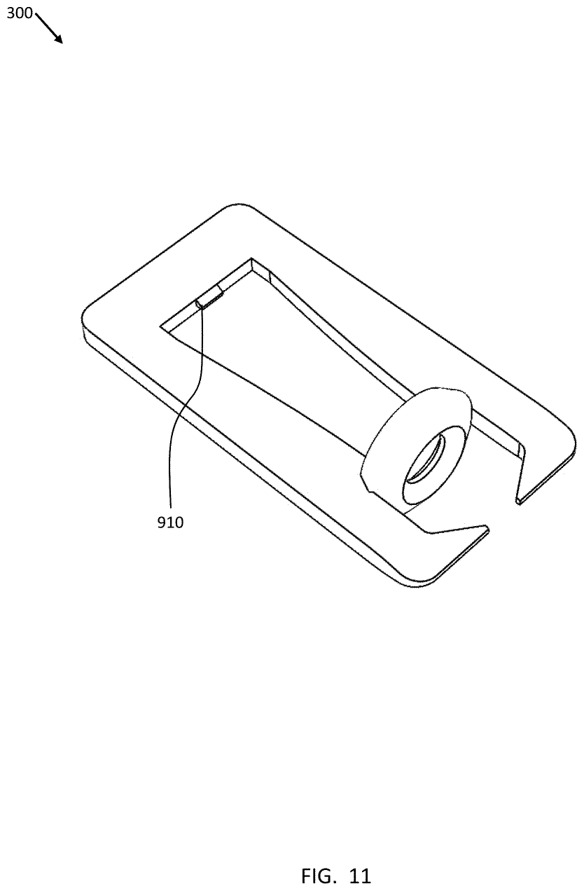

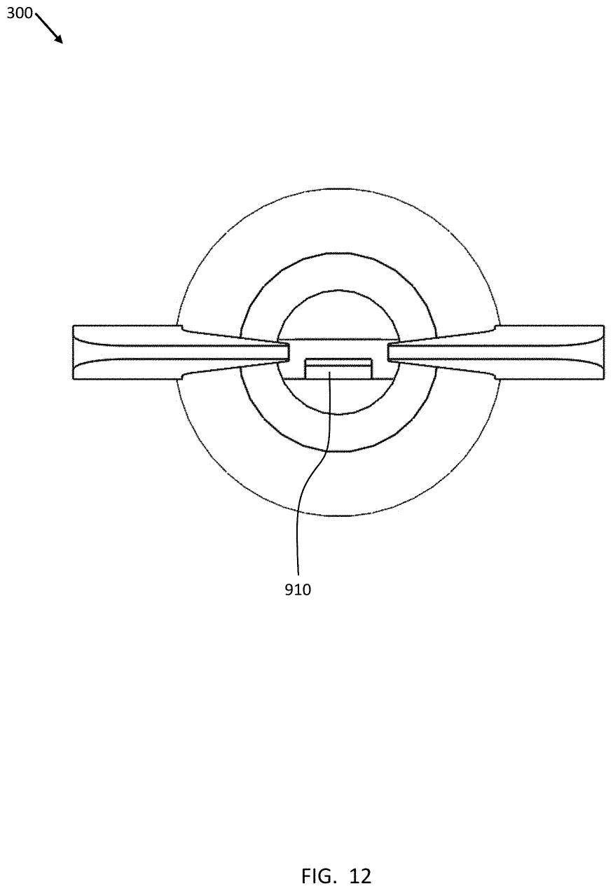

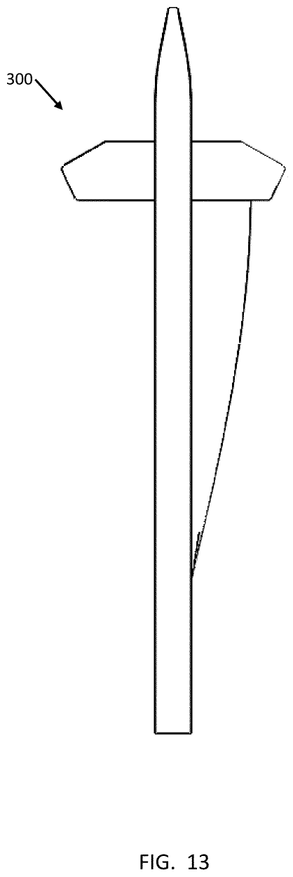

Moreover, FIGS. 10-13 illustrate further three dimensional representations of other aspects of exemplary dispensers described herein (e.g., such as in FIGS. 1-3). For example, FIG. 10 illustrates a view of an exemplary device from above, viewed along the first surface, directed at the proximal surface, and into and through the aperture. FIG. 11 illustrates a view of an exemplary device from above and from the direction of the hyperacuity target(s) into and through the aperture from the direction opposite the proximal surface of the collar. FIG. 12 illustrates a view of an exemplary device from the end of the device past the hyperacuity target(s) and into and through the aperature (from the side opposite the proximal surface). FIG. 13 illustrates a side view of an exemplary device comprising a recess within the first surface.

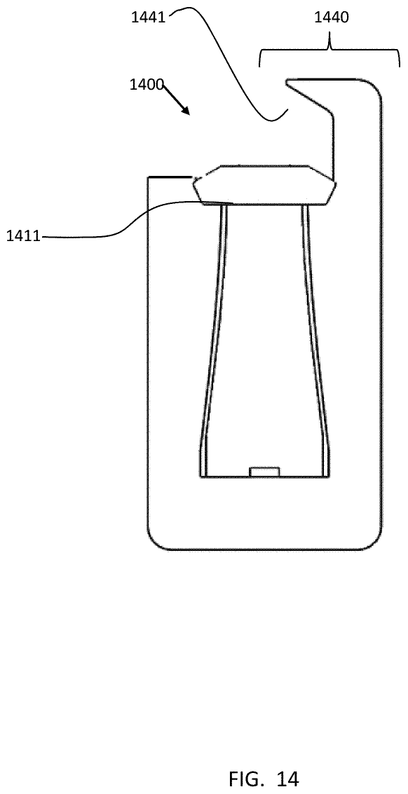

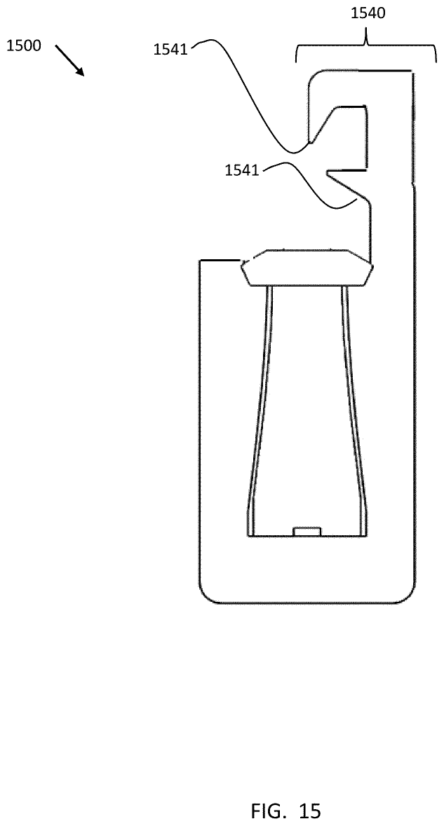

FIG. 14 shows an exemplary dispenser 1400 comprising a single hyperacuity target 1440, such as having a single indication feature 1441(e.g., corresponding to a similar structure as a proximal point in other devices illustrated herein). As shown, in some instances, an upper surface of the single hyperacuity target 1440 is generally parallel with the collar 1411 of the dispenser 1400. FIG. 15 shows an exemplary dispenser 1500 comprising a single hyperacuity target 1540 having two indication features 1541. Alternatively, in some embodiments, the dispenser comprises more than two indication features 1441 1541. As shown, in some instances, an upper surface of a lower hyperacuity target 1540 is generally parallel with the collar 1411 of the dispenser 1400, such as while a left (or right, depending on the side the hyperacuity target is configured) surface of an upper hyperacuity target 1540 is generally perpendicular to the collar 1411 of the third dispenser 1400.

In some embodiments, the dispenser 100, 900, 1400, 1500 comprises a single-use dispenser.

Product tube for precise secretion of a semi-solid product

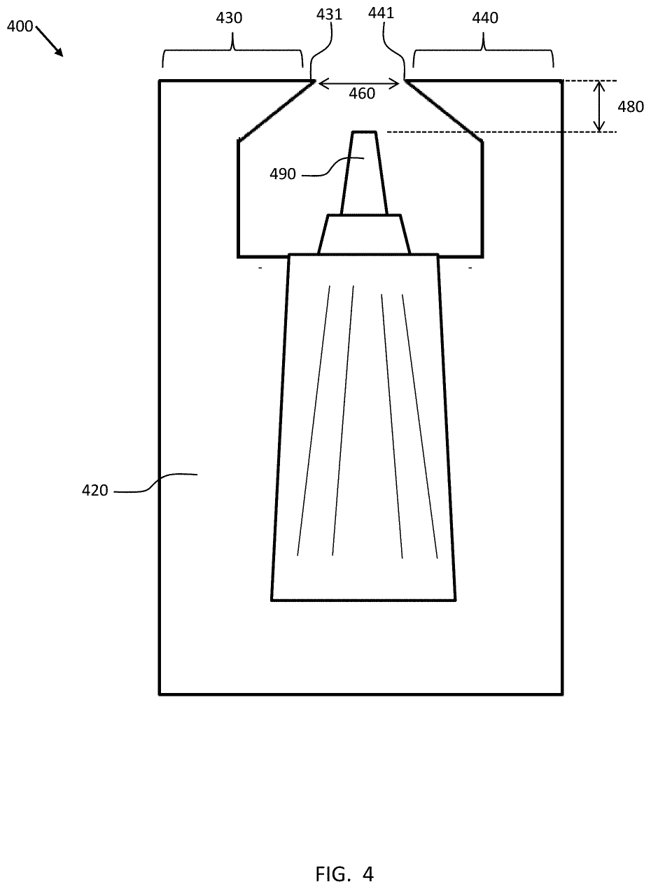

Another aspect provided herein, per FIG. 4, is an exemplary (e.g., semi-solid) product tube 400 for precise secretion of a semi-solid. The semi-solid may comprise an ointment, a cream, or a gel. In some instances, such a product tube is an integrated product comprising a dispenser, such as discussed herein, and a product tube, such as otherwise discussed herein.

In some embodiments, the (e.g., semi-solid) product tube 400 comprises a product tube body 420, a nozzle 490, and at least one hyperacuity target 430, 440. The at least one hyperacuity target 430, 440 may comprise a first hyperacuity target 430 and a second hyperacuity target 440. At least one of the first hyperacuity target 430 and the second hyperacuity target 440 may provide a visual indication corresponding to the dispensing measurement distance and a secreted volume of the semi-solid product. In some embodiments, the first semi-solid product tube 400 comprises a single-use product tube 400.

The product tube body 420 may be configured to contain the semi-solid product. The nozzle 490 may be configured to secrete the semi-solid product from the product tube body 420. The nozzle 490 may have an inner diameter. In some embodiments, the first semi-solid product tube 400 comprises comprising plastic, wood, metal, fiberglass, carbon fiber, resin, or any combination thereof.

In some embodiments, the first hyperacuity target 430 and the second hyperacuity target 440 further extend towards each other. In some embodiments, the first hyperacuity target 430 and the second hyperacuity target 440 further extend towards each other to form a hyperacuity target separation gap 460. The separation gap 460 may be measured between a first proximal point and a second proximal point. The separation gap 460 may be measured as a minimum distance between the first proximal point 431 on the first hyperacuity target 430 and the second proximal point 441 on the second hyperacuity target 440. The separation gap 460 may be measured as a minimum normal distance between the first proximal point 431 on the first hyperacuity target 430 and the second proximal point 441 on the second hyperacuity target 440. The separation gap 460 may be measured as a minimum normal distance between the first hyperacuity target 430 and the second hyperacuity target 440. In some embodiments, the hyperacuity target separation gap 460 is about 2 mm to about 60 mm. In some embodiments, the hyperacuity target separation gap 460 is at least about 2 mm. The at least one hyperacuity target 430, 440 may terminate at a set distance from the nozzle 490 of the product tube to prevent contact between the at least one hyperacuity target 430 440 and the semi-solid product. The separation gap may be designed to prevent contact between the first hyperacuity target 430 and the semi-solid product, the second hyperacuity target 440 and the semi-solid product, or both.

The first hyperacuity target 430 may extend from the nozzle 490 by a dispensing measurement distance 480. The second hyperacuity target 440 may extend from the nozzle 490 by the dispensing measurement distance 480. In some embodiments, the dispensing measurement distance 480 is a greater than a distance from the shoulder of the product tube to a distal point of the dispensing tip. In some embodiments, the dispensing measurement distance 480 is about 1 mm to about 15 mm. In some embodiments, the dispensing measurement distance 480 is about 1 mm to about 7 mm. In some embodiments, the dispensing measurement distance 480 is at least about 1 mm. The dispensing measurement distance 480 may be measured as a maximum distance between the nozzle 490 and a first distal point on the first hyperacuity target 430 or a second distal point on the second hyperacuity target 440. The dispensing measurement distance 480 may be measured as a maximum normal distance between the nozzle 490 and the first distal point on the first hyperacuity target 430 or the second distal point on the second hyperacuity target 440. In some embodiments, the first distal point is the first proximal point 431. In some embodiments, the second distal point is the second proximal point 441. In some embodiments, the dispensing measurement distance 480 corresponds to the secreted volume of the semi-solid product. In some embodiments, the secreted volume corresponds to a viscosity, a melting temperature, a kinematic viscosity, or any combination thereof of the semi-solid product. In some embodiments, the secreted volume (e.g., of any product tube, dispenser, or combination thereof) is about 1 ul to about 10 ul. In some embodiments, the secreted volume is about 1 mg to about 10 mg. In some embodiments, the secreted volume accuracy is about 0.1 ul to about 10 ul. In some embodiments, the secreted volume of the semi-solid product corresponds to the dispensing measurement distance (D) 480 and the inner diameter of the nozzle 490 (ID). In some embodiments, the secreted volume of the semi-solid product is equal to:

$$D \times {π ( \frac{ID}{2} )}^{2}$$

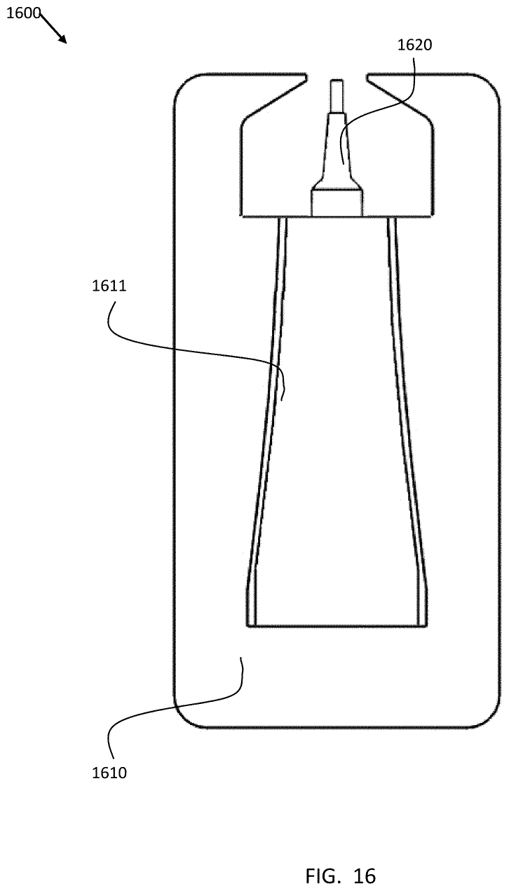

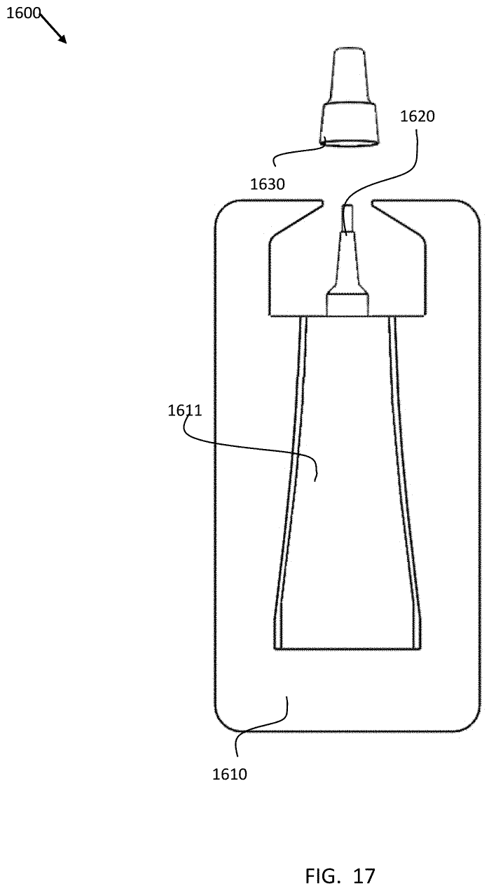

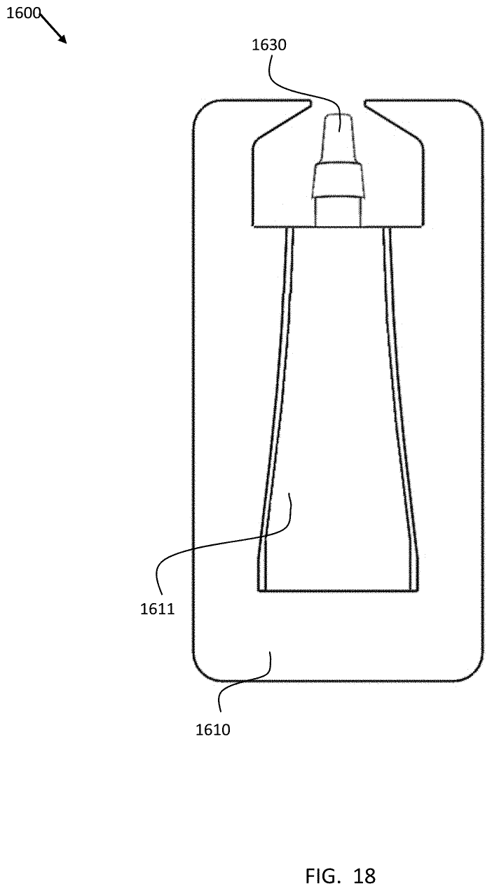

FIGS. 16-20 show illustrations of an exemplary product tube 1600 (e.g., integrated product tube with dispenser and/or product tube input into a dispenser, such as described herein) comprising a first product tube body (or a dispenser body) 1610, a second product tube body 1611, and a nozzle 1620. In some embodiments, such as illustrated in FIGS. 17-18 the second semi-solid product tube 1600 further comprises a cap 1630, which may be placed over the nozzle 1620 when the semi-solid is not being dispensed, such as illustrated in FIG. 18. The cap 1630 may be configured to cover and protect the nozzle 1620.

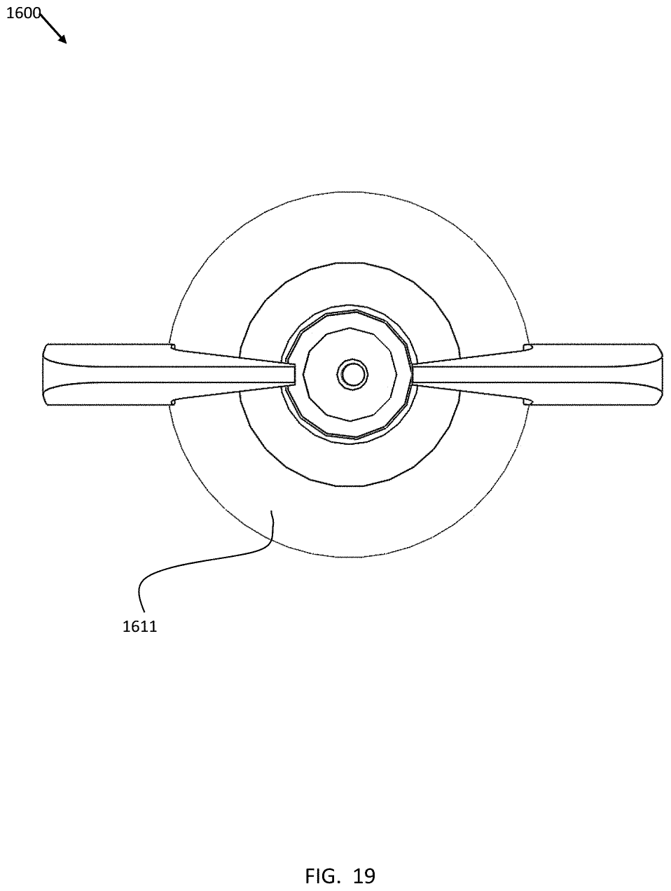

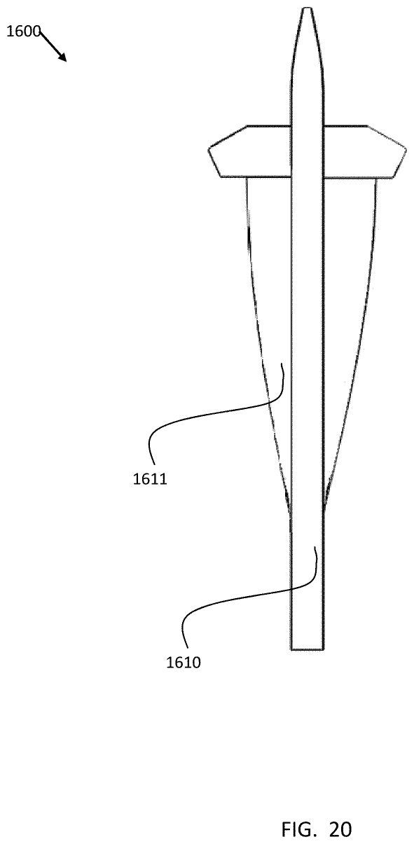

FIG. 19 further illustrates a view of an exemplary (e.g,. integrated) product tube from the end of the product tube past the hyperacuity target(s) and into the nozzle of the product tube. FIG. 20 illustrates a side view of an exemplary product tube integrated with hyperacuity target(s) and first product tube body 1610.

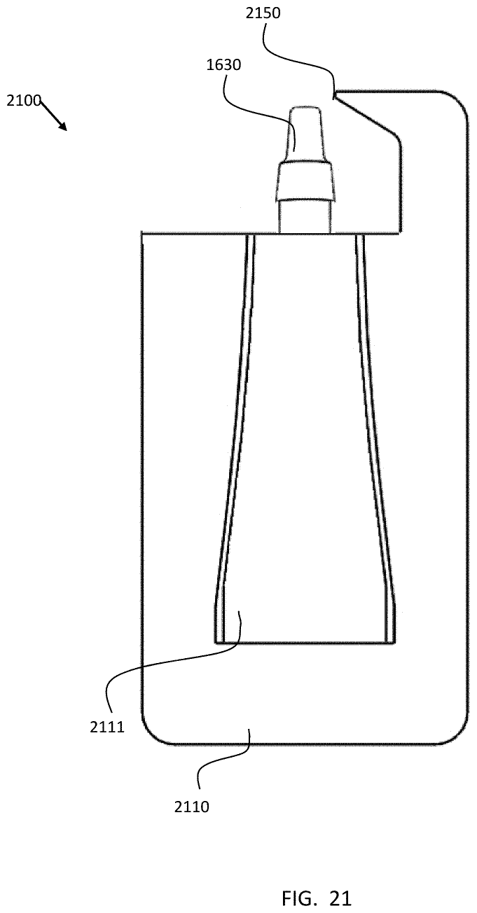

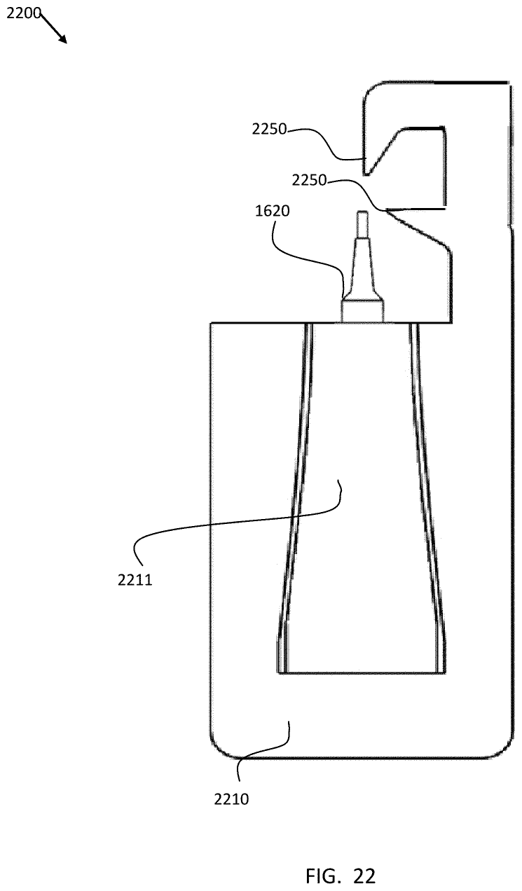

FIG. 21 shows an illustration of an exemplary (e.g., integrated) (e.g., semi-solid) product tube 2100 comprising a first product tube (or dispenser body) body 2110, a second product tube body 2111, and a cap 1630 covering a nozzle, wherein the first product tube body 2110 comprises a single hyperacuity target 2150. FIG. 22 shows an illustration of an exemplary (e.g., integrated) (e.g., semi-solid) product tube 2200 comprising a first product tube body (or dispenser body) 2210, a second product tube body 2211, a nozzle 1620, wherein the first product tube body 2210 comprises two hyperacuity targets 2250.

In some embodiments, the cap 1630 removably attaches to the product tube body 1611. In some embodiments, the cap 1630 removably attaches to the product tube body 1611 though a snap feature, a screw, a press fit, a hook and loop fastener, a tie, a sticker, or any combination thereof.

System for precise secretion of a semi-solid product

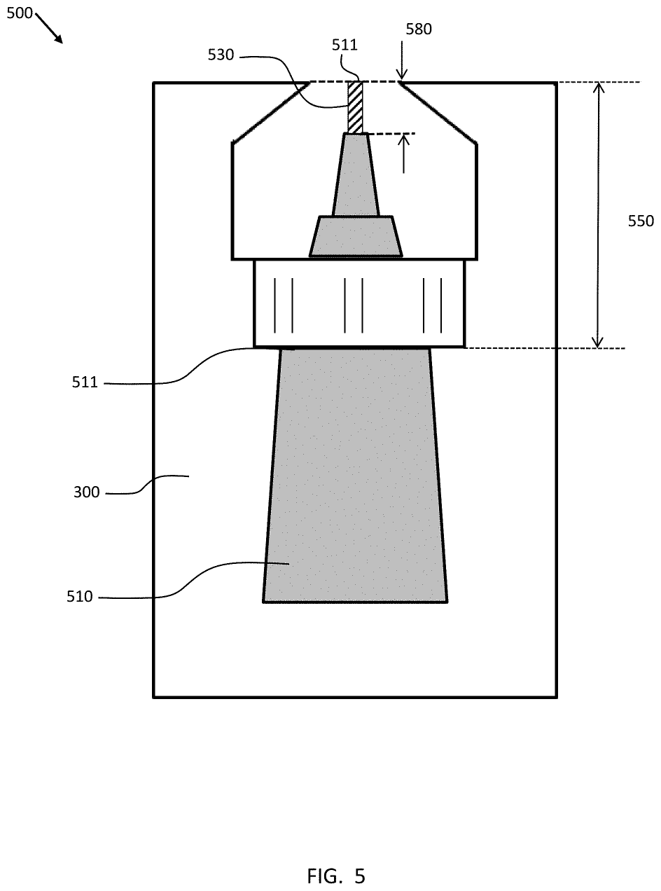

Another aspects provided herein, per FIGS. 5-8, are systems for precise secretion of a semi-solid product.

As shown in FIG. 5, the exemplary first system 500 for precise secretion of a semi-solid product comprises a product tube 510, and a second dispenser 300. In some embodiments, the product tube 510 has a shoulder 511 and a dispensing tip 512. In some embodiments, the product tube 510 further comprises a cap. The semi-solid 530 may comprise an ointment, a cream, or a gel.

The second dispenser 300 may comprise a collar and a first surface comprising a first hyperacuity target and a second hyperacuity target. The collar may comprise an aperture and a proximal surface. The aperture may be configured to accept the dispensing tip 512. The aperture may be configured to accept the cap. The proximal surface may be configured to contact the 512 of the product tube 510.

In some embodiments, the first surface further comprises a cavity configured surround at least a portion of the product tube 510. In some embodiments, the aperture of the collar comprises a threaded feature. In some embodiments, the second dispenser 300 comprises plastic, wood, metal, fiberglass, carbon fiber, resin, or any combination thereof.

At least one of the first hyperacuity target and the second hyperacuity target may provide a visual indication corresponding to the dispensing measurement distance and a secreted volume of the semi-solid product 530.

The first hyperacuity target may extend from the proximal surface 511 by a dispensing offset length 550. The second hyperacuity target may extend from the proximal surface 511 by the dispensing offset length 550. In some embodiments, the dispensing offset length 550 is a greater than a distance from the shoulder of the product tube to a distal point of the dispensing tip. In some embodiments, the dispensing offset length 550 is about 1 mm to about 100 mm. In some embodiments, the dispensing offset length 550 is at least about 1 mm. The dispensing offset length 550 may be measured as a maximum distance between the proximal surface 511 and a first distal point on the first hyperacuity target or a second distal point on the second hyperacuity target. The dispensing offset length 550 may be measured as a maximum normal distance between the proximal surface 511 and the first distal point on the first hyperacuity target or the second distal point on the second hyperacuity target. In some embodiments, the first distal point is the first proximal point. In some embodiments, the second distal point is the second proximal point. In some embodiments, the dispensing offset length 550 corresponds to the secreted volume of the semi-solid product 530. In some embodiments, the secreted volume is about 1 ul to about 10 ul. In some embodiments, the secreted volume is about 1 mg to about 10 mg. In some embodiments, the secreted volume accuracy is about 0.1 ul to about 10 ul. In some embodiments, the secreted volume corresponds to a viscosity, a melting temperature, a kinematic viscosity, or any combination thereof of the semi-solid product 530.

The first hyperacuity target may extend from the nozzle by a dispensing measurement distance 580. The second hyperacuity target may extend from the nozzle by the dispensing measurement distance 580. In some embodiments, the dispensing measurement distance 580 is a greater than a distance 580 from the shoulder of the product tube to a distal point of the dispensing tip. In some embodiments, the dispensing measurement distance 580 is about 1 mm to about 15 mm. In some embodiments, the dispensing measurement distance 580 is at least about 1 mm. The dispensing measurement distance 580 may be measured as a maximum distance 580 between the nozzle and a first distal point on the first hyperacuity target or a second distal point on the second hyperacuity target. The dispensing measurement distance 580 may be measured as a maximum normal distance 580 between the nozzle and the first distal point on the first hyperacuity target or the second distal point on the second hyperacuity target. In some embodiments, the first distal point is the first proximal point. In some embodiments, the second distal point is the second proximal point. In some embodiments, the dispensing measurement distance 580 corresponds to the secreted volume of the semi-solid product 530. In some embodiments, the secreted volume corresponds to a viscosity, a melting temperature, a kinematic viscosity, or any combination thereof of the semi-solid product 530. In some embodiments, the secreted volume is about 1 ul to about 10 ul. In some embodiments, the secreted volume is about 1 mg to about 10 mg. In some embodiments, the secreted volume accuracy is about 0.1 ul to about 10 ul. In some embodiments, the secreted volume of the semi-solid product 530 corresponds to the dispensing measurement distance 580 (D) and the inner diameter of the nozzle (ID). In some embodiments, the secreted volume of the semi-solid product 530 is equal to:

$$D \times {π ( \frac{ID}{2} )}^{2}$$

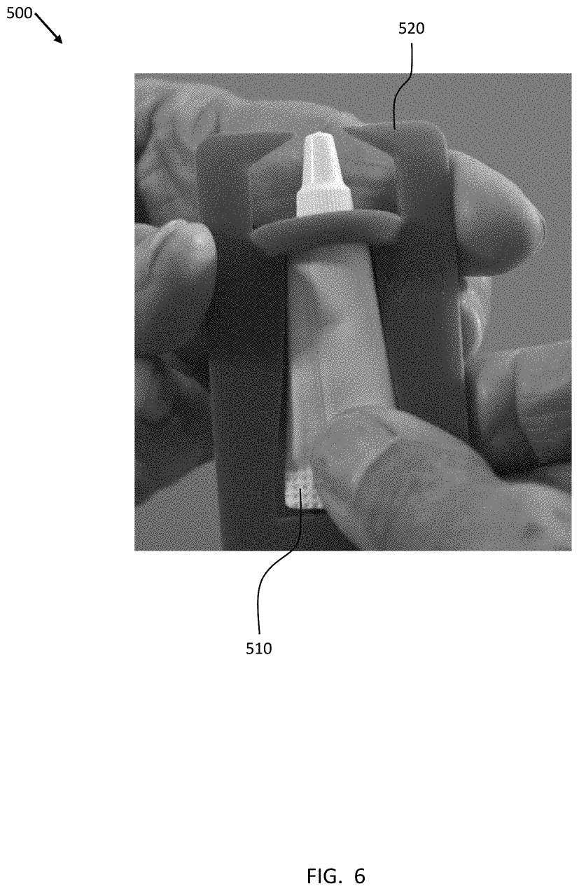

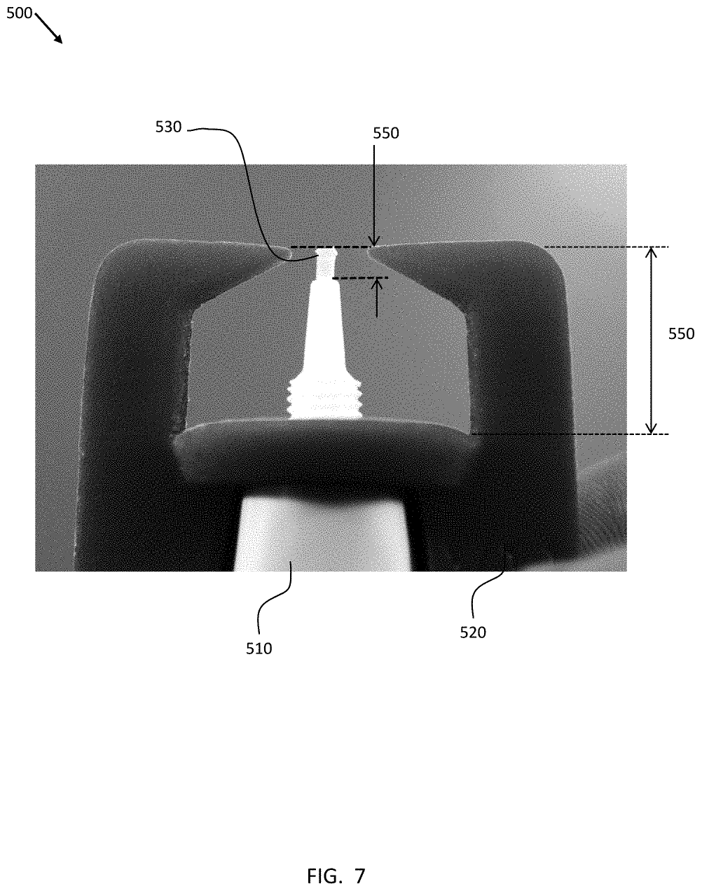

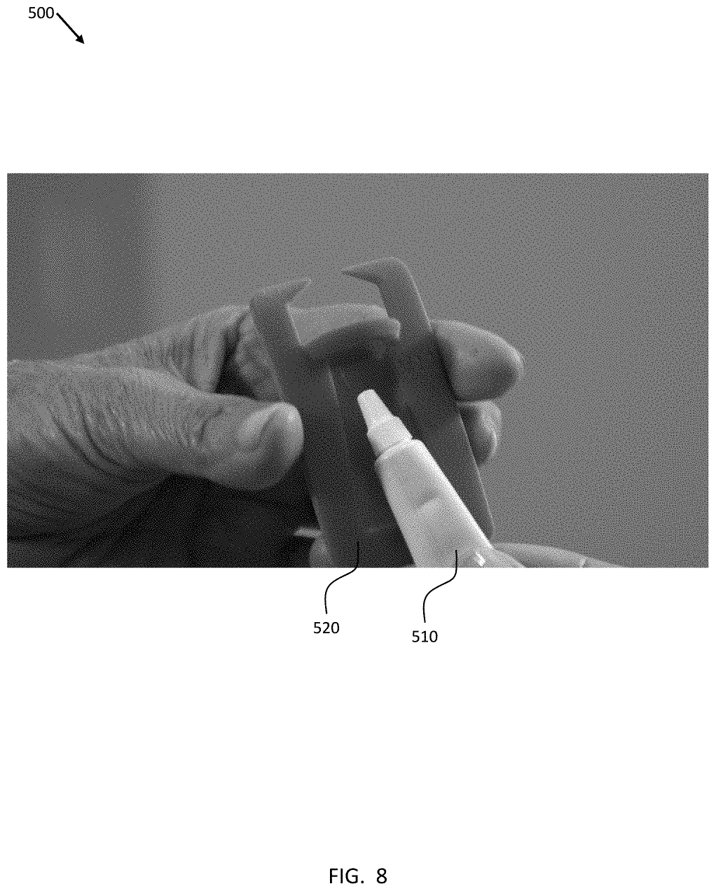

FIGS. 6-7 show images of the exemplary system 500 for precise secretion of a semi-solid product. FIG. 8 shows an image of the insertion of the product tube 510 into the second dispenser 300 of the exemplary system 500 for precise secretion of a semi-solid product.

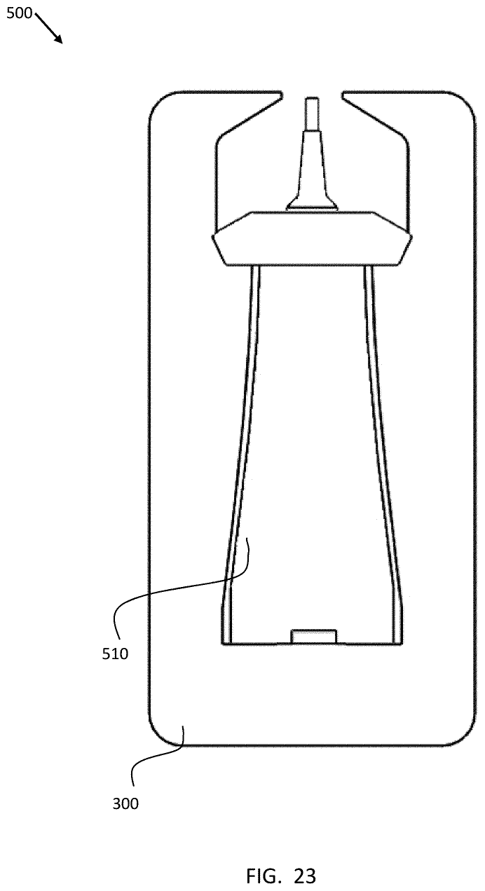

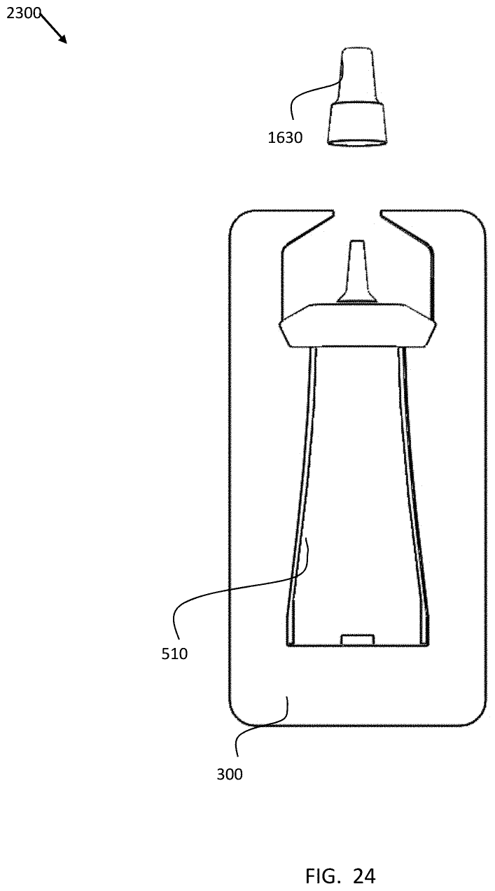

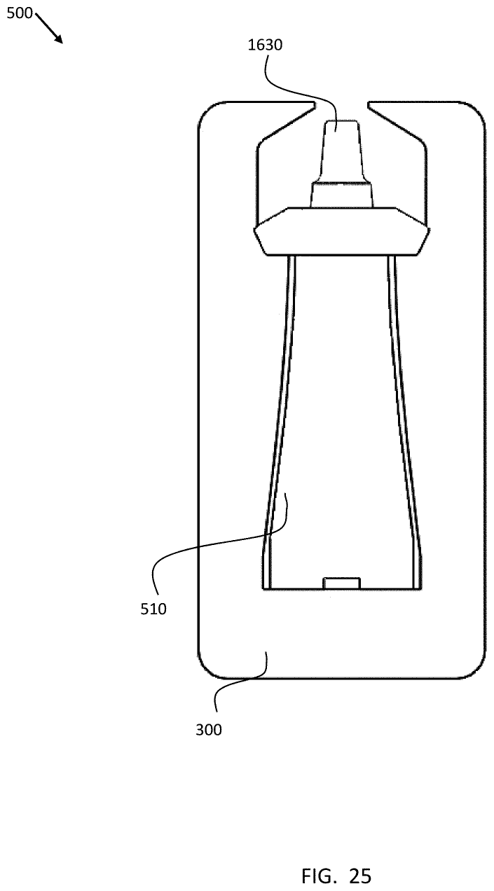

FIGS. 23-25 display illustrations of the exemplary first system 500 2300 comprising a semi-solid product dispenser 300 and a product tube 510. Per FIGS. 24-25, in some embodiments, the system 500 further comprises a cap 1630. The cap 1630 may be configured to cover and protect a nozzle of the product tube 510.

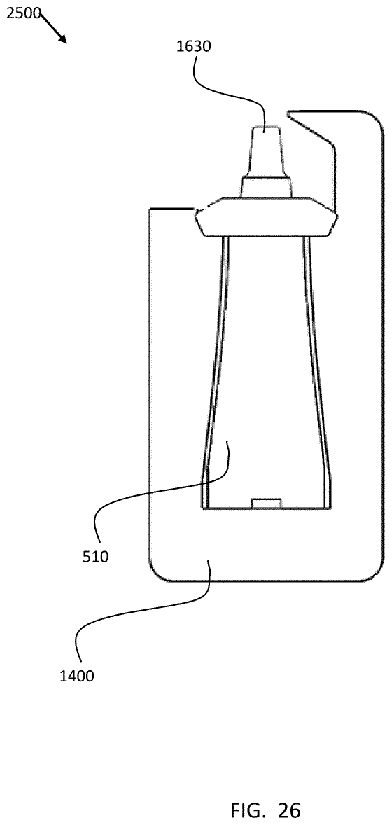

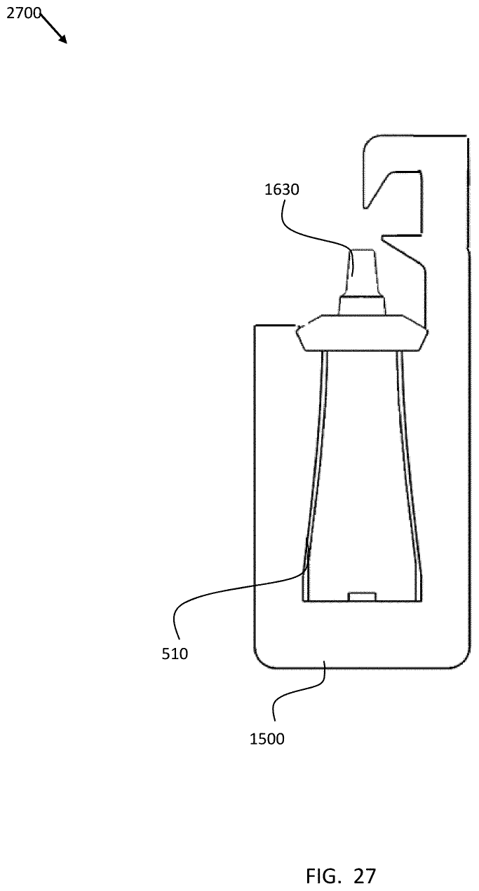

FIG. 26 is a front view illustration of an exemplary second system 2600 comprising the third solid product dispenser 1400, a product tube 510, and a cap 1630 covering the nozzle of the a product tube 510. FIG. 27 is a front view illustration of an exemplary system 2700 comprising the fourth solid product dispenser 1500, a product tube 510, and a cap 1630 covering the nozzle of the a product tube 510.

In some embodiments, the cap 1630 removably attaches to the product tube 510. In some embodiments, the cap 1630 removably attaches to the product tube 510 though a snap feature, a screw, a press fit, a hook and loop fastener, a tie, a sticker, or any combination thereof.

Semi-solid product dispensing kits

Another aspect provided herein is a kit comprising two or more product tubes, each product tube having a product and a dispenser. Each of the two or more semi-solid product tubes may comprise the same semi-solid product or different semi-solid products. In some embodiments, the kit further comprises an applicator. The applicator may comprise at least one of a Q-tip, a swab, a tissue, and a brush. In some embodiments, the kit further comprises an at least one of an instruction manual, a mirror and a wipe. The wipe may comprise one or more of a tissue, a towel, a paper, and a q-tip and a brush.

Methods for precise secretion of a semi-solid product

Provided herein is a first method for precise secretion of a semi-solid product comprising: providing a product tube; inserting the dispensing tip of the product tube within the aperture of the dispenser; aligning the shoulder of the product tube against the proximal surface of the dispenser; and compressing the product tube to secrete the semi-solid product until the semi-solid product is level with a proximal portion of the first hyperacuity target, a proximal portion of the second hyperacuity target, or both.

In some embodiments, the method further comprises removing the secreted semi-solid product from the product tube. In some embodiments, removing the secreted semi-solid product from the product tube comprises removing the secreted semi-solid product from the product tube with an applicator or a finger. In some embodiments, the method further comprises applying the semi-solid product to a patient. In some embodiments, the product tube further comprises a cap, and wherein the method further comprises removing the cap. In some embodiments, the cap is removed before inserting the dispensing tip of the product tube within the aperture of the dispenser. In some embodiments, the cap is removed after inserting the dispensing tip of the product tube within the aperture of the dispenser. In some embodiments, the method further comprises replacing the cap onto the product tube after removing the secreted semi-solid product from the product tube. In some embodiments, the semi-solid product generally maintains its cylindrical shape once it is secreted from the nozzle of the product tube.

Also provided herein is a second method for precise secretion of a semi-solid product comprising: providing a product tube; and compressing the product tube to secrete the semi-solid product until the semi-solid product is level with a proximal portion of the first hyperacuity target, a proximal portion of the second hyperacuity target, or both.

In some embodiments, the method further comprises removing the secreted semi-solid product from the product tube. In some embodiments, removing the secreted semi-solid product from the product tube comprises removing the secreted semi-solid product from the product tube with an applicator or a finger. In some embodiments, the method further comprises applying the semi-solid product to a patient. In some embodiments, the product tube further comprises a cap, and wherein the method further comprises removing the cap. In some embodiments, the method further comprises replacing the cap onto the product tube after removing the secreted semi-solid product from the product tube. In some embodiments, the semi-solid product generally maintains its cylindrical shape once it is secreted from the nozzle of the product tube.

### TERMS AND DEFINITIONS

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “generally” refers to an amount that is identical to the stated amount or near the stated amount by 15 degrees, 10 degrees, 5, degrees, or 1 degree, including increments therein.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

### EXAMPLES

### Example 1

### Dispensing Repeatability

The relationship between ointment weight and volume was measured at 5 microliter=4.5 mg. As such to dispense 4.5 mg of an exemplary ophthalmic ointment from a tube tip having an internal diameter of 1.27 mm, the dispenser was configured to extrude a 5 microliter sample having a length of about 4 mm.

To evaluate the repeatability of an ophthalmic product amount (mg) dispensed from the dispenser, two subjects dispensed an amount of 4 mg of a product from a product tube using the dispenser described herein. The dispensed amount was weighed per Tables 1 and 2 below:

TABLE 1

| Subject | Amount Dispensed (mg) |
|---|---|
| 1 | 4.6 |
| 1 | 4.3 |
| 1 | 4.6 |
| 1 | 4.8 |
| 1 | 4.1 |
| 1 | 4.6 |
| 1 | 5.9 |
| 1 | 4.7 |
| 1 | 5 |
| 2 | 5.3 |
| 2 | 4.6 |
| 2 | 4.6 |
| 2 | 4.5 |
| 2 | 4.3 |
| 2 | 4.5 |
| 2 | 4.5 |

TABLE 2

| Subject | 1 | 2 |
|---|---|---|
| Average | 4.73 | 4.61 |
| Standard Deviation | 0.51 | 0.32 |
| % Difference | 10.77 | 6.90 |

What is claimed is:

1. A dispenser system for precise secretion of a semi-solid product comprising:
a) a product tube comprising:
i) a tube body configured to contain the semi-solid product;
ii) a nozzle configured to secrete the semi-solid product from the tube body; and
b) a dispenser body comprising a hyperacuity target extending beyond the nozzle by a dispensing measurement distance;
wherein the at least one hyperacuity target provides a visual indication corresponding to the dispensing measurement distance and a secreted volume of the semi-solid product.

2. The dispenser system of claim 1, wherein the product tube further comprises
a) a shoulder, and
b) a dispensing tip at the distal end of the nozzle.

3. The dispenser system of claim 2, wherein the dispenser body further comprises
a) a collar comprising:
i) an aperture configured to accept the dispensing tip; and
ii) a proximal surface configured to contact the shoulder of the product tube
b) a first surface generally perpendicular to the proximal surface, wherein the first surface comprises the hyperacuity target extending from the proximal surface by a dispensing offset length.

4. The dispenser system of claim 3, wherein the dispenser body comprises a first hyperacuity target and a second hyperacuity target.

5. The dispenser system of claim 4, wherein the first hyperacuity target and the second hyperacuity target extend towards each other to form a hyperacuity target separation gap between a first proximal point on the first hyperacuity target and a second proximal point on the second hyperacuity target.

6. The dispenser system of claim 5, wherein the hyperacuity target separation gap is about 1 mm to about 60 mm.

7. The dispenser system of claim 5, wherein the hyperacuity target separation gap is at least about 1 mm.

8. The dispenser system of claim 4, wherein a distal edge of at least one of the first hyperacuity target and the second hyperacuity target are generally perpendicular to the proximal surface.

9. The dispenser system of claim 4, wherein the distal edge of the first hyperacuity target and the distal edge of the second hyperacuity target are generally parallel or collinear.

10. The dispenser system of claim 3, wherein the dispensing offset length is greater than a distance from the shoulder of the product tube to a distal point of the dispensing tip.

11. The dispenser system of claim 3, wherein the dispensing offset length is about 1 mm to about 50 mm.

12. The dispenser system of claim 3, wherein the dispensing offset length is at least about 1 mm.

13. The dispenser system of claim 1, wherein the product tube further comprises a cap.

14. The dispenser system of claim 3, wherein the aperture is further configured to accept the cap.

15. The dispenser system of claim 3, wherein the aperture has a diameter of about 5 mm to about 50 mm.

16. The dispenser system of claim 3, wherein the aperture has a diameter of at least about 5 mm.

17. The dispenser system of claim 3, wherein the first surface further comprises a cavity configured surround at least a portion of the product tube.

18. The dispenser system of claim 3, wherein the aperture of the collar comprises a threaded feature.

19. The dispenser system of claim 1, wherein the product tube and the dispenser body form an integrated product.

20. The dispenser system of claim 1, wherein the product tube and dispenser body form discrete components of the system.
